(19) United States
(12) Patent Application Publication
(10) Pub. No.: US 2024/0416423 A1
(43) Pub. Date: Dec. 19, 2024

(54) ADDITIVE MANUFACTURING WITH CONTRAST AGENTS

(71) Applicant: Hewlett-Packard Development Company, L.P., Spring, TX (US)

(72) Inventors: Clara REMACHA CORBALAN, Sant Cugat del Valles (ES); Virginia PALACIOS CAMARERO, Sant Cugat del Valles (ES); Kristopher J. ERICKSON, Palo Alto, CA (US)

(21) Appl. No.: 18/704,875

(22) PCT Filed: Oct. 29, 2021
(86) PCT No.: PCT/US2021/057251

§ 371 Date: Apr. 25, 2024

(51) Int. Cl. B22F 10/80; B22F 10/28; B28B 1/00; B29C 64/153; B29C 64/386; B29K 23/00; B29K 75/00; B29K 77/00; B29K 105/00; B29K 105/16; B29K 505/00; B29K 509/00; B29L 31/00; B33Y 10/00; B33Y 50/00; B33Y 70/10; B33Y 80/00

(52) U.S. Cl. CPC B22F 10/80; B22F 10/28; B28B 1/001; B29C 64/153; B29C 64/386; B33Y 10/00; B33Y 50/00; B33Y 70/10; B33Y 80/00; B29K 2023/12; B29K 2075/00; B29K 2077/00; B29K 2105/0005; B29K 2105/162; B29K 2505/00; B29K 2509/00; B29L 2031/7546

(57) ABSTRACT

In an example, a method includes defining, for an interior portion of an object to be generated in additive manufacturing, an imaging characteristic in a radiation environment. In some examples the method further includes deriving object model data for generating the interior portion of the object by determining an amount of a first contrast agent and an amount of a second contrast agent to deposit such that the object generated according to the object model data comprises the defined imaging characteristic apparent in an image of at least a part of the object comprising the interior portion captured in the radiation environment. The first contrast agent and the second contrast agent may have different imaging characteristics in the radiation environment.

### BACKGROUND

Additive manufacturing techniques may generate a three-dimensional object through the solidification of a build material, for example on a layer-by-layer basis. In examples of such techniques, build material may be supplied in a layer-wise manner and the solidification method may include heating the layers of build material to cause melting in selected regions. In other techniques, chemical solidification and/or binding methods may be used.

### BRIEF DESCRIPTION OF DRAWINGS

Non-limiting examples will now be described with reference to the accompanying drawings, in which:

FIG. 1 is an example of a method for obtaining object model data for use in generating an object in additive manufacturing;

FIG. 2 is an example of an apparatus for obtaining an image indicative of attenuation of radiation of an object generated using additive manufacturing;

FIG. 3 is an example method of generating an object according to object model data by additive manufacturing;

FIG. 4 is an example of an apparatus;

FIG. 5 is another example of an apparatus; and

FIG. 6 is an example machine-readable medium associated with a processor.

### DETAILED DESCRIPTION

Additive manufacturing techniques may generate a three-dimensional (3D) object through the solidification of a build material. In some examples, the build material is a powder-like granular material, which may for example be a plastic, ceramic or metal powder and the properties of generated objects may depend on the type of build material and the type of solidification mechanism used. Build material may be deposited, for example on a print bed and processed layer by layer, for example within a fabrication chamber. According to one example, a suitable build material may be Polyamide materials (e.g., PA12, PA11), Thermoplastic Polyurethane (TPU) materials, Thermoplastic Polyamide materials (TPA), Polypropylene (PP) and the like.

In some examples, selective solidification is achieved through directional application of energy, for example using a laser or electron beam. In other examples, at least one print agent may be selectively applied to the build material, and may be liquid when applied. For example, a fusing agent (also termed a ‘coalescence agent’ or ‘coalescing agent’) may be selectively distributed onto portions of a layer of build material in a pattern derived from data representing a slice of a 3D object to be generated. The data may be derived from a digital or data model of the object, e.g. object model data providing a data, or virtual, model of an object to be generated. The fusing agent may have a composition which absorbs energy such that, when energy (for example, heat) is applied to the layer, the build material to which it has been applied heats up, coalesces and solidifies, upon cooling, to form a slice of the 3D object in accordance with the pattern.

A suitable fusing agent may be an ink-type formulation comprising carbon black. Such a fusing agent may comprise any or any combination of an infra-red light absorber, a near infra-red light absorber, a visible light absorber and a UV light absorber.

In addition to a fusing agent, in some examples, a print agent may comprise a coalescence modifier agent, which acts to modify the effects of a fusing agent for example by reducing or increasing coalescence or to assist in producing a particular finish or appearance to an object, and such agents may therefore be termed detailing agents. In some examples, detailing agent may be used near edge surfaces of an object being generated to reduce coalescence. A coloring agent, for example comprising a dye or colorant, may in some examples be used as a fusing agent or a coalescence modifier agent, and/or as a print agent to provide a particular color for the object.

As noted above, additive manufacturing systems may generate objects based on structural design data. This may involve a designer determining a data model of an object to be generated, for example using a computer aided design (CAD) application. The model may define the solid portions of the object. To generate a 3D object from the model using an additive manufacturing system, the model data can be processed to define slices or parallel planes of the model. Each slice may define a portion of a respective layer of build material that is to be solidified or caused to coalesce by the additive manufacturing system.

Medical imaging and radiotherapy can be used to diagnose and treat patients by the application of radiation to a patient. Examples of medical imaging include x-ray imaging, CT (computed tomography), PET (positron emission tomography), fluoroscopy, MRI (magnetic resonance imaging) and ultrasound. As used herein, radiation refers to the transmission of energy in the form of waves, such as electromagnetic radiation or acoustic radiation. Some methods of medical imaging utilise non-ionising radiation such as MRI or ultrasound, whereas other methods use ionising radiation such as x-ray or CT. Radiation can also be used to treat patients in radiotherapy, for example using radionuclide therapy, brachytherapy, or external beam radiation therapy.

For the accuracy of the images, and therefore diagnoses, the apparatus used for medical imaging should be calibrated accurately. Moreover, as in some examples ionising radiation may be used in treatment of patients, the apparatus should be calibrated accurately, because applying an incorrect dose of radiation or applying radiation to the wrong part of the body may harm the patient.

Objects referred to as ‘phantoms’ may be used in calibration of medical imaging and radiotherapy apparatus, and for training of medical personnel. A phantom in this context is an object with a particular shape and imaging characteristic(s) (such as attenuation) which may be imaged or irradiated.

Attenuation is a measure of the loss of intensity of radiation as it passes through a material. For example, when a beam of x-rays is incident on a low attenuation material, such as soft body tissues (e.g. fat, muscle) a large portion of the x-ray radiation passes through the material, and a small portion is absorbed. In contrast, when a beam of x-rays is incident on a high attenuation material, such as hard body tissue (e.g. bone) a significant proportion of the radiation is absorbed. The amount of transmitted radiation is measured and compared with the amount of incident radiation to obtain the attenuation.

Attenuation is a property of an object and depends on the shape, size and attenuation coefficients of the object, wherein the attenuation coefficient is a property of the material. In particular the attenuation depends on the attenuation coefficient and the path length of the radiation through the object. Attenuation is dependent on the path length through the material and the attenuation coefficient for a homogeneous object. The attenuation properties of a material may, for example, be measured using the Hounsfield scale (HU). In the Hounsfield scale, distilled water is defined to have a value of 0 HU and air is defined to have a value of −1000 HU. Attenuation measured on the Hounsfield scale can be obtained from the linear attenuation coefficient of a material according to the equations:

$$H U = 1000 \times \frac{μ - {μ}_{w}}{{μ}_{w} - {μ}_{α}}$$

wherein μ is the linear attenuation coefficient of the material, μ_{w} is the linear attenuation coefficient of water and μₐ is the linear attenuation coefficient of air. The linear attenuation coefficient is defined as

$$μ = - \frac{1}{Φ} \frac{d Φ}{d z}$$

wherein Φ is the radiant flux and z is the path length of the beam.

Different types of body tissue may have different attenuation properties, therefore a phantom may comprise portions which have different attenuations to ensure the apparatus is calibrated across the range of attenuations of different tissue types. A phantom may be designed for use with a particular type or energy of radiation. For example, a phantom may be designed to have a particular attenuation when irradiated with x-rays or ultrasound. Some phantoms may be suitable for use with different types of apparatus, for example a phantom may be designed to be suitable for calibration of x-ray imaging apparatus and CT apparatus.

Some examples of phantoms may be designed to mimic the human body, or a portion thereof. Such phantoms are referred to as anthropomorphic phantoms and may be constructed from various materials which have similar attenuation to tissue of the human body. Other phantoms may not be designed to mimic the human body, for example simple geometry phantoms may comprise materials with different attenuations arranged in a relatively simple geometry. For example, they may comprise a cylinder having a particular attenuation with holes into which other modules (e.g. cylinders) having another particular attenuation can be inserted. These phantoms may be expensive to produce, especially anthropomorphic phantoms which often include complex geometries and manual assembly.

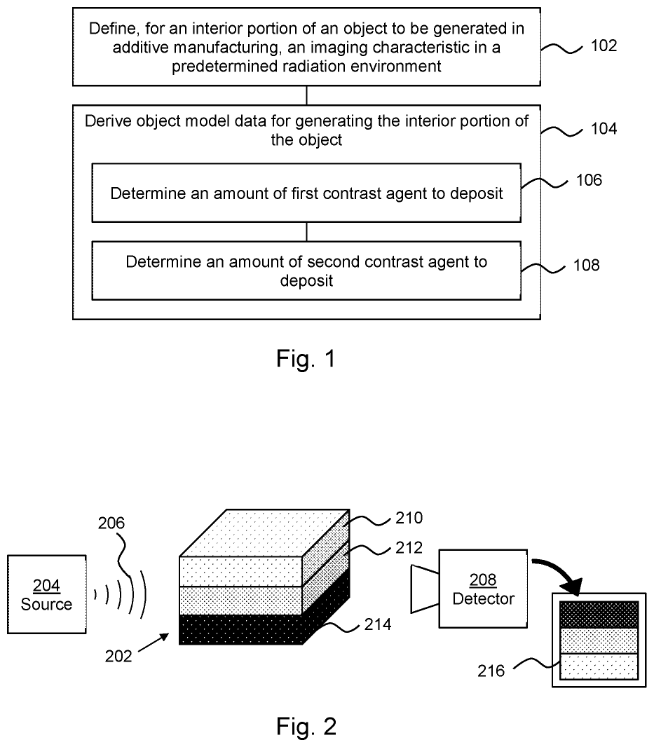

FIG. 1 is an example of a method, which may comprise a method for deriving object model data for use in generating an object in additive manufacturing. In this example the method is carried out at least in part by processing circuitry, which may comprise at least one processor.

The method comprises, in block 102, defining, for an interior portion of an object to be generated in additive manufacturing, an imaging characteristic in a radiation environment. Defining the imaging characteristic may comprise associating an intended attenuation level, or an intended contrast level, with the interior portion. In some examples the radiation environment is a predetermined radiation environment. The radiation environment may define a type and/or energy of radiation. For example in medical imaging or treatment, the radiation environment may refer to the radiation used for imaging or treating a patient. For example x-ray radiation with an energy of greater than 5 keV or greater than 10 keV may be used in radiography. The imaging characteristic may be a property which is measured during imaging. For example, if the radiation environment is an x-ray radiation environment for radiography, the associated imaging characteristic may be an attenuation. In other examples, the radiation environment may be an MRI radiation environment and the associated imaging characteristic may be spin polarization of 15 hydrogen atoms, which are visible as contrast differences in a captured MRI image. In some examples, other object portions may be associated with an imaging characteristic.

As used herein, an interior portion may refer to a portion of the object which is not normally visible due to being located internally within the object and/or to any portion of an object which is behind another object portion in a direction (or an intended direction) of imaging. In other words, radiation reaching an interior portion will have passed through at least one other object portion before reaching the interior portion (albeit that the other object portion may also be associated with an imaging characteristic as set out herein). An interior portion may refer to a portion of the object which is not exposed or is further at least partially (and in some examples, fully) surrounded by material of the object. For 25 example, the interior portion may be a portion which is within a fused portion of build material or on an inner surface of a fused portion.

The method comprises, in block 104, deriving object model data for generating at least the interior portion of the object, and may comprise deriving object model data for generating other portion(s) of the object, or the object as a whole. The object model data may define the size and shape of the object to be generated. The object model data may for example comprise a Computer Aided Design (CAD) model, and/or may for example be a STereoLithographic (STL) data file or a 3D Manufacturing Format (3MF) data file. The object model data may comprise a representation of the object, for example as a plurality of voxels or a mesh model.

In some examples the object model data may be derived from a digital or a data model representing the object to be generated, for example from a virtual model of the object. Such a model may be from a CAD model designed to represent the object or obtained from another physical object. For example the object model data may be derived from an image (or images) representing an object, for example captured using a similar medical imaging method which is intended to be used with the generated object. For example, an original object (e.g. a human or animal patient) may be imaged using radiography or MRI, and the image(s) of the original object processed to obtain the model representing the object to be generated.

The object model data may also comprise a description of the defined imaging characteristic. For example, when the object model data is derived from a radiography image, the object model data may comprise attenuation data describing the intended attenuation in the interior portion of the object to be generated.

The object model data may define amounts and locations to deposit agents when generating the object in additive manufacturing. In some examples different amounts of agents may be associated with different regions of the object. For example in regions which are intended to be solidified to form part of the object, fusing agent may be deposited. Detailing agent may be deposited near edge surfaces of the object. Other agents, such as coloring agents may additionally be deposited. In addition to the above agents, the object model data may define the locations and amounts of contrast agents to be deposited when generating the object. The contrast agents may modify a property of the finished object, such as a contrast level when the object is imaged, for example in radiography the contrast level may be a measure of the attenuation of the region of the object.

Where a 3D image (or a series of 2D images) has been received, this may be processed to obtain data at a resolution of an intended 3D printer. For example, the images may have a separation of Yμm, whereas a 3D printing layer may have a height of Xμm. When Y is smaller than X, images may be combined to provide an imaging characteristic value for a 3D printing voxel. For example, if three images relate to one layer to be generated in additive manufacturing, the average imaging characteristic value of three corresponding pixels in those images may be determined as an imaging characteristic value for an additive manufacturing pixel. While the height of a layer has been considered here, the same could be true for a width or a depth of voxel, and the appropriate combination may depend on the angle of imaging and on the intended orientation of object generation. In other examples, when Y is larger than X, each image may provide an imaging characteristic value for more than one layer. In still further examples, in which the voxel dimensions in additive manufacturing are configurable, these may be configured based on the resolution of an image and/or the separation between images.

Block 104 further comprises, in block 106, determining an amount of a first contrast agent to deposit and, in block 108, determining an amount of a second contrast agent to deposit. The amounts of the first and second contrast agents are determined such that an object generated according to the object model data comprises the defined imaging characteristic, such that it is (at least approximately) apparent in an image of at least a part of the object comprising the interior portion captured in the radiation environment. As further described below, it may be intended to image the object in each of a plurality of different radiation environments and it may be intended that the object exhibits, at least approximately, a defined imaging characteristic in each of these environments. In such examples, a plurality of imaging characteristics may be defined.

A contrast agent may be an agent which is associated with a change in the way radiation passes through a generated object, and the first and second contrast agents may affect this to a different degree to one another. In some examples, the contrast agent may reduce the amount of radiation which passes through the generated object.

When generating the object according to the object model data, the object model data may define the amount of each contrast agent and/or an amount of other print agents such as fusing agent to be deposited, for example within a region of the object or within each voxel of the object. Therefore, when generating the object, some regions of the object may comprise no contrast agent, some regions may comprise one contrast agent and other regions may comprise both contrast agents, depending on the intended imaging characteristics of each region.

In some examples, the contrast agent may itself have the properties of a fusing agent (i.e. is capable of absorbing energy to cause build material to heat, melt and fuse) and/or an amount of a separate fusing agent may be specified for portions of the object which it is intended to fuse.

The amount of fusing agent specified may take account of the thermal contribution of the contrast agent. For example, where contrast agent has some energy absorbing properties, an amount of fusing agent specified may be reduced in a region in which contrast agent is to be applied compared to an analogous region in which contrast agent is not to be applied. By contrast, in some examples, a contrast agent may have a cooling effect on the build operation, and in such examples an amount of fusing agent specified may be increased in a region in which contrast agent is to be applied compared to an analogous region in which contrast agent is not to be applied.

In addition, a change in imaging characteristics may be associated with an amount of fusing agents and this may be taken into account when specifying an amount of the first and second contrast agent.

By using more than one type of contrast agent, an object may be generated which has more accurate imaging characteristics, is able to obtain a wider range of imaging characteristics, or which has different imaging characteristics when imaged using different imaging methods. Therefore, the first contrast agent and the second contrast agent have different imaging characteristics in the radiation environment. For example, a contrast agent may increase the attenuation of the build material. In such an example, the first contrast agent may be associated with a first change in attenuation when a predetermined volume thereof is applied to build material, and the second contrast agent may be associated with a second, different, change in attenuation when the same predetermined volume thereof is applied to build material. In another example, a contrast agent may provide a particular change in the MRI response of the build material. In such an example, the first contrast agent may be associated with a first change in response when a predetermined volume thereof is applied to build material, and the second contrast agent may be associated with a second, different, change in response when the same predetermined volume thereof is applied to build material.

In some examples, the first contrast agent may provide a relatively large change in the imaging characteristic and the second contrast agent may provide a relatively small change in the imaging characteristic. Therefore, the first contrast agent may be used to achieve a wide range of imaging characteristic levels and the second contrast agent may provide more precise control of the imaging characteristic level.

In some examples the object is a medical imaging phantom and may be intended to represent a human or animal body, or a portion thereof. An image may be obtained of the body (portion) and the image used to derive the object model data for generating the medical imaging phantom. For example the image may be obtained using medical imaging methods, such as x-ray imaging, CT (computed tomography), PET (positron emission tomography), fluoroscopy, MRI (magnetic resonance imaging) or ultrasound and may comprise at least one two-dimensional or at least one three-dimensional image.

The medical imaging phantom may be intended to simulate a generic human or animal body, or a portion thereof. The phantom may be a generic phantom, in that it is intended to represent a body or a portion of a body without any identifying or unique features. In some examples, the object model data used to generate the object may be modified to introduce an abnormality (or a modelled abnormality), such as an indication of a disease.

In some examples the phantom may be intended to represent a specific patient or a patient with a particular condition, such as a tumour, broken bone, cardiopathies, organ malfunctions or any trauma condition. In some examples the image used to derive the object model data comprises image data representing a plurality of tissue types, wherein at least one tissue type is an abnormal tissue and wherein deriving a plurality of zones comprises associating each zone with a tissue type. In such examples, the body which is imaged may have the condition. In such examples, the phantom may be used for treatment or diagnoses of the particular patient, for example in simulating application of radiation in radiotherapy, or for calibrating a particular imaging machine using the phantom as a known baseline. For example, this may help in more accurately tracking the progress of a condition or treatment, even when different imaging apparatus and/or technologies are used. In other examples, the phantoms may be generally used in calibration of apparatus used in medical imaging or radiotherapy, as medical training models.

Medical training models are objects which represent a body and are used in training for example for surgery. For example, a medical training model may represent a particular pathology and may be used to train or practice treating that pathology in surgery. Some types of surgery may be performed while being imaged, for example using x-ray imaging, CT imaging, MRI, ultrasound imaging or using any other suitable medical imaging. For example, vascular interventions may be performed under x-ray imaging. Therefore, a medical imaging phantom may be used for training for such surgery since it comprises attenuation properties intended to mimic the attenuation properties of the body. Medical imaging phantoms which are intended to be used as medical training models may be constructed so that their physical properties mimic those of a patient. For example, the materials may be selected to have similar properties to those of the relevant body tissue, for example a material may be selected which has a similar elasticity, flexibility, strength as a particular tissue (e.g. skin, muscle or fat). In such examples, selecting additive manufacturing attributes for each zone may be based on other intended physical properties, such as elasticity, flexibility or strength, in addition to the attenuation characteristics.

In some examples the phantom may not be representative of a (portion of a) human or animal body and instead may comprise an abstract shape and properties. For example, the phantom may be a regular shape such as a sphere or a cube or may be any other suitable shape such as a cuboid or an ellipsoid, and may comprise interior regions with different predetermined properties, such as different attenuations. Such phantoms may be useful in calibrating medical imaging or treatment devices.

In some examples the object is a radiation shield. A radiation shield may be designed to protect a person's body, or a portion thereof, from radiation by having an attenuation sufficient to shield the person from radiation. In some examples, the shield may be designed to have an attenuation above a threshold attenuation for a particular type, or multiple types, of radiation. For example the radiation shield may be designed such that it has an attenuation sufficient to reduce the amount of radiation reaching the person's body to a safe level when they are exposed to a radiation source. The radiation shield may be shaped to fit the person. For example the radiation shield may be generated with a shape to fit a particular person's body, for example a three-dimensional model of the (part of) the person's body may be obtained and the object model data representing the radiation shield to be generated may be determined based on the model of the (part of the) person's body.

In some examples, when the imaging characteristic is contrast in MRI, the first and/or the second contrast agents comprise at least one of: gadolinium, a metal salt, a metallogenic compound, a metal oxide or metal nanoparticles of any or any combination of: gadolinium, copper, manganese or iron. In further examples the first and/or the second contrast agents comprise at least one of a metal salt, a metallogenic compound, a metal oxide or metal nanoparticles of any or any combination of: manganese, silver, gold, cobalt, nickel, zinc, molybdenum, europium, ytterbium, dysprosium. In some examples the first and/or the second contrast agents comprise perfluorinated organic compounds.

When the imaging characteristic is an attenuation of x-ray radiation, the contrast agent(s) may comprise an element with a high atomic number (Z), for example the contrast agent(s) may comprise an element with an atomic number greater than or equal to 40. In some examples the contrast agent(s) comprise a copper salt or silver nanoparticles.

For example the copper salt may be a hydrated metal salt and comprise an anion selected from the group consisting of hydroxidecarbonate, sulfate, nitrate, acetate, formate, borate, chloride, bromide, and combinations thereof. An example salt is hydrated copper nitrate. The contrast agent comprising the copper salt may further comprise a carrier liquid comprising at least one surfactant and water, wherein the at least one copper salt is present in an amount of at least 5 wt % in the contrast agent based on the total weight of the contrast agent. An example of such an agent is described in US 2021/0178466.

In some examples a contrast agent comprises silver nanoparticles. The silver nanoparticles may comprise the individual element silver or comprise an alloy thereof. The nanoparticles may have a Z-average particle size of from about 20 nanometres to about 70 nanometres as determined by dynamic light scattering, among other possibilities. For example, the silver nanoparticles can comprise metal particles having a Z-average particle size of from about 20 nanometres to about 60 nanometres. As another example, the at least one conductive particulate can comprise metal particles having a Z-average particle size of from about 30 nanometres to about 50 nanometres. Such a metallic agent may comprise energy absorbing qualities such that it may contribute to, or cause, fusion in build material when a layer of build material having the agent applied thereto is irradiated. An example of such an agent is described in US 2020/0269501.

As described above, phantoms may be designed to simulate various tissue types which have a wide range of imaging characteristics. Print agents, such as contrast agent, may be deposited with a range of coverage amounts describing the quantity of agent deposited and may be described in terms of a contone level, which may vary on an arbitrary scale between a minimum and a maximum coverage amount. When a contrast agent is deposited to achieve a particular contrast level, the amount of contrast agent may be variable in discrete levels, which may correspond to drop sizes of contrast agent and/or a number of drops of contrast agent within a zone, for example the levels may be contone levels which range from 0 to 256, wherein the maximum contone level 256 corresponds to one drop per printed pixel. Therefore, in order to accurately reproduce the wide range of imaging characteristics (such as attenuation of x-ray radiation or spin polarization of hydrogen atoms visible as contrast differences in MRI imaging), the first contrast agent may be selected to provide a relatively large change in the imaging characteristic to provide a coarse control of the contrast level and to achieve higher contrast levels and the second contrast agent may be selected to provide a relatively small change in the imaging characteristic to provide fine control of the contrast level to accurately reproduce the intended contrast level and to achieve relatively low contrast levels. Therefore depositing the first contrast agent may provide a relatively larger change in the imaging characteristic (for example, per unit volume of an applied liquid agent) and depositing the second contrast agent may provide a relatively smaller change in the imaging characteristic (for example, per the same unit volume of an applied liquid agent). In other words, the change in imaging characteristic caused by the first and second contrast agents per unit volume of agent per unit area, volume or mass of build material may be different.

In some examples the imaging characteristic is an attenuation. Therefore, each unit of the first contrast agent which is deposited may provide a large change in attenuation whereas each unit of the second contrast agent may provide a relatively small change in attenuation. In some examples the attenuation is attenuation of x-ray radiation. For example lung tissue may have an attenuation in the range of −950 HU to −550 HU, fat may have an attenuation in the range −100 HU to −80 HU and bone may have an attenuation above 50 HU for x-ray radiation. Therefore, in order to create a portion of an object which simulates lung tissue, only the first contrast agent may be used. To create a portion of an object which simulates fat a small amount of the second contrast agent may be used and an amount of the first contrast agent may additionally be used to accurately target the intended attenuation. In order to create a portion of the object which simulates bone a relatively large amount of the second contrast agent may be used, and optionally an amount of the first contrast agent may be used to accurately target the intended attenuation. For example, this may be achieved using different compositions in the contrast agent and/or varying a concentration of an attenuating component between the different agents.

In some examples, it may be intended to generate an object which has intended imaging characteristics when imaged by different types of imaging methods/radiation environments. In such examples, different imaging characteristics may be defined for the object portion. For example it may be intended to create a medical imaging phantom which may be used with MRI and radiography (x-ray or CT imaging). Therefore, the first contrast agent, and the amount thereof, may be selected to provide an intended imaging characteristic when the object is imaged using a first method in a first radiation environment and the second contrast agent, and the amount thereof may be selected to provide an intended imaging characteristic when the object is imaged using a different second imaging method in a second radiation environment. For example the amount of the first contrast agent may be selected to provide an intended x-ray attenuation and the amount of the second contrast agent may be selected to provide an intended magnetic resonance imaging contrast.

For example, the first contrast agent may comprise at least one of a copper salt or silver nanoparticles and the second contrast agent may comprise gadolinium.

FIG. 2 shows an object 202 and apparatus which may be used to obtain an image indicative of attenuation of radiation by the object 202. The object 202 is generated according to the method described in relation to FIG. 1. The apparatus comprises a source 204 which emits radiation 206. In some examples the source 204 is a source of x-rays, for example an x-ray tube, and the radiation 206 is x-ray radiation suitable for imaging patients. The radiation 206 is incident on the object 202. Some of the radiation 206 passes through the object and is detected by the detector 208, which is located on the opposite side of the object 202 to the source 204. The detector 208 may be any means suitable for detecting the radiation, for example x-ray radiation may be detected using x-ray film, image plates or flat panel detectors. In other examples the source 204 may emit a different type of radiation, for example it may comprise a radioactive source such as ¹⁹²Ir, ¹³⁷Cs or ⁶⁰Co to provide gamma rays, a transducer to produce ultrasound or a linear accelerator. In some examples MRI apparatus may be used to measure the image of the object 202.

In the example, the object 202 comprises three portions: an upper portion 210, a middle portion 212 and a lower portion 214. In this example the upper portion 210 has a low attenuation, the middle portion 212 has an intermediate attenuation and the lower portion 214 has a high attenuation. The upper portion 210 was generated without application of any contrast agent to build material (although fusing agent was applied thereto in order to achieve the low attenuation). The middle portion 212 was generated by applying fusing agent and the first contrast agent in order to achieve the intermediate attenuation. The lower portion 214 was generated using fusing agent and the second contrast agent in order to achieve the high attenuation. In some examples, both the first and second contrast agents may be used when generating portions with a high attenuation. The second contrast agent may be used to achieve the high attenuation while the first contrast agent may provide fine control of the achieved attenuation.

When the first object 202 is irradiated or illuminated with radiation 206 from the source 204, some of the incident radiation passes through the first object 202 and is detected by the detector 208. A small portion of the radiation is absorbed by the upper portion 210 because it has a low attenuation. Therefore, a relatively large proportion of the radiation which passes through the upper portion 210 is detected by the detector 208. Conversely, a large portion of the radiation which is incident on the lower portion 214 is absorbed by the material, and therefore a relatively small proportion of the radiation which is incident on the lower portion 214 is not detected by the detector 208. In this way the detector 208 is able to measure the attenuation of different parts of the first object 202.

In some examples the detector 208 may be located on the same side of the object as the source 204, and rather than detecting transmitted waves, it detects reflected waves. For example, in ultrasound imaging, reflected ultrasound waves are detected by a detector 208 located on the same side, an often integrated with, the source 204. In such examples, the source 204 may be a piezoelectric transducer.

In other examples the source 204 and detector 208 may have different relative positions. For example, the source 204 may be placed inside the first object 202. Such a configuration may be used in, or to simulate, PET (positron emission tomography), which comprises injecting a radioactive tracer into a patient's body.

An image 216 may be created based on the measured attenuations. In this example, in the image 216, the attenuation level is encoded in the greyscale value such that regions with high attenuation are represented by lighter shading and regions with lower attenuation are represented by darker shading. In some examples a plurality of images of the object 202 may be obtained, for example a plurality of two-dimensional images may be obtained, from which a three-dimensional representation of the object may be determined.

In some examples, the image 216 may be automatically partitioned into ‘zones’ wherein each zone is associated with a range of attenuation values. In some examples, the image may for example be processed before it is divided into zones, for example reducing the information therein by reducing the resolution thereof, simplifying contours, removing portions thereof, and/or removing noise and/or artefacts.

The attenuation of the material may depend on the physical or geometrical structure of the generated object. Therefore, in order to produce the object 202 in additive manufacturing with the intended properties, other additive manufacturing attributes may be varied, such as the physical structure (e.g. a lattice structure, or microstructure) in addition to the amount of contrast agents. For example, solidifying built material to have a lattice structure (e.g. a honeycomb-like structure) may provide a reduced attenuation when compared to a fully solidified block of build material, whereas use of one or two contrast agents may provide an increased attenuation when compared to a fully solidified block of build material without the contrast agent.

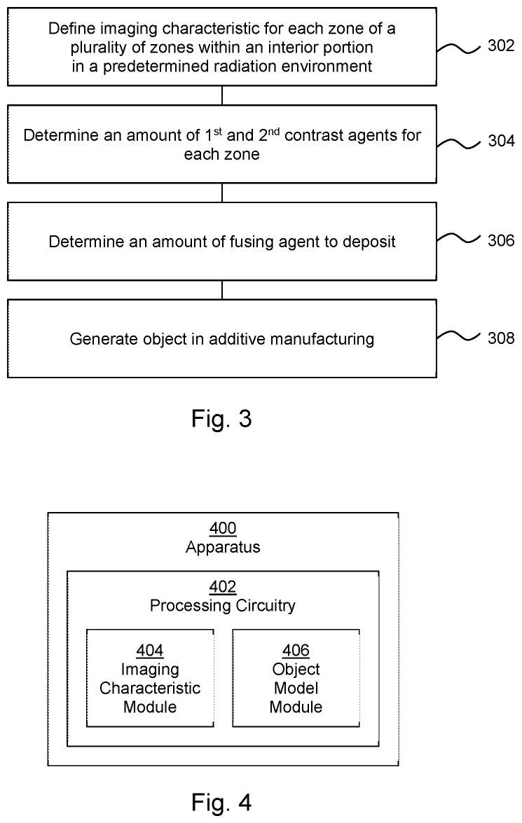

FIG. 3 provides an example of the method of FIG. 1. As discussed in greater detail below, blocks 302 and 304 may provide an example of the method of blocks 102 and 104 described in relation to FIG. 1.

Block 302 comprises defining an imaging characteristic for each zone of a plurality of zones within an interior portion of an object to be generated using additive manufacturing. In some examples each zone corresponds to a region which is intended to have the same imaging characteristic, for example a constant attenuation. In examples wherein the object is a medical imaging phantom, a zone may correspond to a region which is intended to represent a particular type of tissue, for example bone, and therefore may have a shape corresponding to a bone and the imaging characteristic may be determined to be an attenuation corresponding to that of bone for example in the range above 50 HU for x-ray radiation. Imaging characteristics may also be defined for other portions, such as exterior portions, of the object to be generated.

In some examples each zone may correspond to a voxel of the object model data representing the object to be generated and each voxel in the model may be associated with an imaging characteristic. In an example wherein the object is a medical imaging phantom comprising a region representing bone, a plurality of voxels may be defined wherein their combined shape represents the shape of a bone and the imaging characteristic associated with each voxel in this region corresponds to the attenuation of bone i.e. above 50 HU for x-ray radiation.

Blocks 304 and 306 comprise deriving the object model data, and may be an example of block 104 of FIG. 1. Block 304 comprises determining an amount of the first contrast agent and an amount of the second contrast agent for each zone. The amount of contrast agent associated with each zone may be determined based on the defined imaging characteristic for each zone, for example the amount of each contrast agent may be determined such that when the object is generated according to the object model data the object has the intended imaging characteristics in each zone. For example, if the defined imaging characteristic is an attenuation value, the amount of the first contrast agent and the second contrast agent may be determined to be the amount of each contrast agent which results in that attenuation when the object is generated.

Deriving the object model data further comprises, in block 306, determining an amount of fusing agent to deposit to cause coalescence of a build material in additive manufacturing by absorbing energy applied to the build material. The fusing agent may be an agent which is deposited in addition to the contrast agents, wherein the fusing agent absorbs energy to cause coalescence of the build material. The contrast agents may also absorb some energy, however the fusing agent may be an agent which absorbs a larger portion of energy than the contrast agents. Furthermore, in some examples, the fusing agent may not comprise any additives which are intended to affect the imaging characteristics. Deriving the object model data may further comprise determining amounts of other agents to deposit, for example detailing agents which may reduce or increase coalescence or assist in producing a particular finish or appearance to an object. In some examples agents may be deposited which alter the appearance of the object, for example coloring agents, such as inks, dyes or colorants, may be defined within the object model data for deposition during generation of the object. In examples in which the contrast agent(s) do absorb enough energy to cause coalescence of the build material, the amount of fusing agent may be zero. In examples in which the contrast agent(s) contribute to energy absorption, the amount of fusing agent may be less than in examples where fusing agent is used without contrast agent, and may depend on the amount of contrast agent to be deposited. In examples in which the contrast agent(s) may have a cooling effect or otherwise inhibit fusing, the amount of fusing agent may be greater than in examples where fusing agent is used without contrast agent, and may again depend on the amount of contrast agent to be deposited.

Block 308 comprises generating the object in additive manufacturing according to the derived object model data. In order to generate the object, print agent may be selectively deposited onto portions of build material. For example, a fusing agent may be deposited in areas which are intended to be solidified to generate the object and a detailing agent may be deposited in regions surrounding the regions intended to be solidified in order to prevent ‘over fusing’ or fusing in areas around the object. Additionally, the detailing agent may be deposited in regions intended to be solidified for temperature control. The contrast agents may be deposited within areas which are intended to be solidified to provide the intended imaging characteristics.

Generating the object using additive manufacturing may comprise obtaining data describing which portions of build material print agent is to be deposited upon, based on the derived object model data representing the object to be generated. Print agent coverage amounts referred to herein may be the print agent coverage amounts to be deposited in these portions, for example a fusing agent coverage amount may refer to the fusing agent coverage amount which is to be deposited in a region intended to be solidified, a detailing agent coverage amount may refer to the detailing agent coverage amount which is to be deposited in a region which is not intended to be solidified and first and second contrast agent coverage amounts may refer to the amount of each contrast agent which is to be deposited to provide the defined imaging characteristics. In some examples, detailing agent is deposited in proximity to regions in which fusing agent is applied, for example about the periphery of a layer of the object being formed, rather than all regions of a layer of build material which are not intended to be solidified. In some examples, detailing agent may be dispensed onto a region of build material which is intended to be solidified. The print agent coverage amounts may for example be defined as an area coverage, that is the volume of printing agent to be deposited per unit area, or as a percentage coverage, that is, the percentage of an area which is intended to be covered with the print agent. In some examples, it may be defined as a contone level. The locations to which print agent drops are applied and/or the amount and/or size of such drops may be determined according to an intended coverage, for example using halftoning techniques and the like.

In other examples, other additive manufacturing techniques may be used to generate the object.

FIG. 4 is an example apparatus 400, which may be used in some additive manufacturing operations, for example in deriving object model data for use in additive manufacturing to generate an object such as a medical imaging phantom. The apparatus 400 comprises processing circuitry 402, the processing circuitry 402 comprising an imaging characteristic module 404 and an object model module 406. In some examples, the processing circuitry 402 may carry out any or any combination of blocks 102 to 108 of FIG. 1, or any combination of blocks 302 to 308 of FIG. 3.

In this example, in use of the apparatus 400, the imaging characteristic module 404 is to obtain, for at least an interior portion of an object to be generated in additive manufacturing, a predetermined image contrast level for when the object is imaged in a predetermined radiation condition. The predetermined image contrast level may correspond to the defined imaging characteristic described above and may for example be an attenuation, when the predetermined radiation condition comprises x-ray radiation for use in x-ray imaging or CT, or may correspond to a MRI environment, and may for example be defined, or may be obtained from a memory, or over a network or the like.

In this example, in use of the apparatus 400, the object model module 404 is to derive object model data for use in generating at least the interior portion of the object. The object model data specifies an amount of a first contrast agent and an amount of a second contrast agent to be deposited during generation of the object to provide the predetermined image contrast level apparent in an image of at least a part of the object comprising the interior portion captured when the object is imaged in the predetermined radiation condition. The first contrast agent has a first imaging characteristic, and the second contrast agent has a second imaging characteristic when imaged in the predetermined radiation condition.

In some examples, the object model data may comprise a plurality of zones, and may define an amount of the first contrast agent and the second contrast agent to be deposited in each zone during generation of the object. The first and second contrast agents may differ in that the first contrast agent provides larger change in the contrast level than the second contrast agent when a predetermined amount (e.g. volume) of each agent is applied to the build material, for example the first contrast agent may contain a larger concentration of the material which affects the contrast level. Therefore, the first contrast agent may be used to achieve relatively higher contrast levels and the second contrast agent may be used to provide lower and/or more precise changes in contrast level. The first and second contrast agents may be used within the same zone, with the first contrast agent providing a high contrast level and the second contrast agent providing precise control of the contrast level.

In other examples the first contrast agent and the second contrast agent may be used to control different imaging characteristics, for example the first contrast agent may be used to provide intended contrast levels when the object is imaged using x-ray radiation and the second contrast agent may be used to provide intended contrast levels when the object is imaged using MRI. In such examples the first and second contrast agent may contain different materials to provide these different properties, for example a contrast agent for controlling contrast when imaged using x-rays may comprise a copper salt or silver nanoparticles, whereas a contrast agent for controlling contrast in MRI may comprise gadolinium.

In some examples, the method may comprise determining amounts of further contrast agents. For example several contrast agents may be used to provide different contrast levels when imaged using a first method and further contrast agents may be used to provide different contrast levels when imaged using a different second imaging method. For example the first and second contrast agents may provide the contrast levels when imaged using x-ray radiation and a third and fourth contrast agent may provide contrast levels when imaged using MRI, wherein the second and fourth contrast agents may provide more precise control of the contrast level then the first and third agents respectively.

In some examples the radiation condition of the radiation environment describes a type of radiation and/or an energy of the radiation. For example, the type of radiation may be x-ray radiation and the energy may define the energy of the x-ray radiation. In other examples the radiation environment may be a MRI radiation environment.

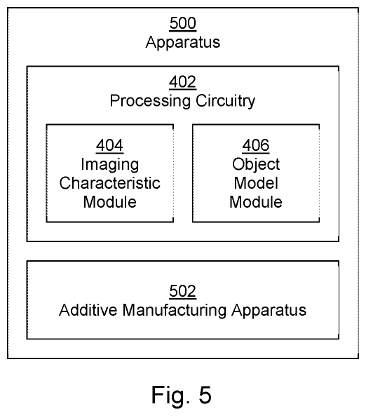

FIG. 5 shows an example of an apparatus 500 comprising processing circuitry 402 which includes the imaging characteristic module 404 and the object model module 406 of the apparatus of FIG. 4. In addition, the apparatus 500 further comprises additive manufacturing apparatus 502. In use of the apparatus 500, the additive manufacturing apparatus 502 is to generate the object based on the derived object model data.

The additive manufacturing apparatus 502 may generate objects in a layer-wise manner by selectively solidifying portions of layers of build material. The selective solidification may in some examples be achieved by selectively applying print agents, for example through use of ‘inkjet’ liquid distribution technologies, and applying energy, for example heat, to each layer using the plurality of fusing energy sources. In some examples, control instructions are determined from the derived object model data modelling the object. For example, the object model data may be ‘sliced’ into slices corresponding to each layer to be generated in additive manufacturing, the portions of the layer which are to be solidified may be identified within the slice, and control instructions generated therefrom. The control instructions may describe where print agent should be placed on a layer of build material in order to generate a layer of the object. For example print material coverage amounts may be determined as outlined above, and then the placement of drops of print agents may be determined using halftoning techniques or the like to provide a determined print agent coverage amount.

In use of the apparatus 500, energy may be provided by fusing energy sources to cause the build material to which fusing agent has been applied to fuse. The additive manufacturing apparatus 500 may comprise other additional components not shown herein, for example a fabrication chamber, at least one print head for distributing print agents, a build material distribution system for providing layers of build material and the like.

The apparatus 400, 500 may, in some examples, carry out at least one of the blocks of FIG. 1 or FIG. 3. In other examples, other types of additive manufacturing may be used, such as fused deposit modelling, directed energy techniques such as laser sintering, stereolithography, use of binding or curing agents, or the like.

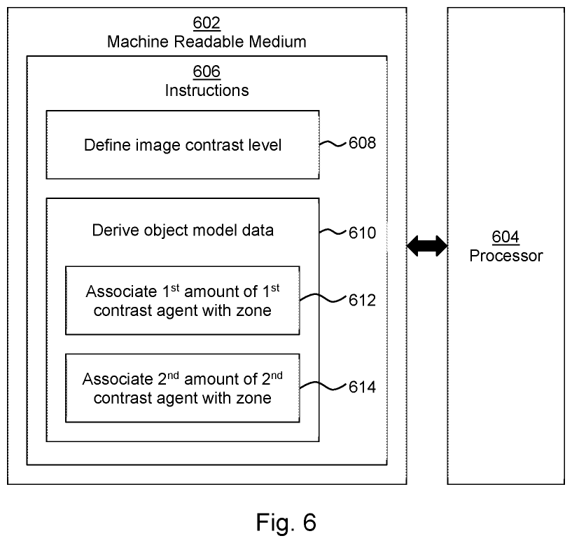

FIG. 6 shows an example of a tangible machine readable medium 602 in association with a processor 604. The machine readable medium 602 stores instructions 606 which, when executed by the processor 604 cause the processor to carry out actions.

In this example, the instructions 606 comprise instructions 608 to cause the processor 604 to define, for an interior portion of an object to be generated in additive manufacturing, an image contrast level in a predetermined radiation environment. The contrast level may for example correspond to an attenuation level in radiography (i.e. x-ray or CT imaging) or contrast level in MRI. The defined image contrast levels may correspond to properties the generated object is intended to have. For example when the object is a medical imaging phantom the object may be intended to have a particular shape and intended image contrast levels, which may be based on a three-dimensional model representing the object. The three-dimensional model may be generated based on an image, or multiple images, obtained using a medical imaging method. For example the image(s) may be obtained using radiography, MRI or any other suitable method. Image contrast levels may also be defined for other object portions, such as external object portions.

In this example, the instructions 606 comprise instructions 610 to cause the processor 604 to derive object model data for generating the interior portion of the object (and may derive object model data for generating other object portions, or the object as a whole). In some examples when an object is generated according to the object model data the image contrast level is apparent in an image captured of the object in the predetermined radiation environment of at least a part of the object comprising the interior portion.

The instructions 610 to derive the object model data comprise, for each of a plurality of zones within the interior portion, instructions 612 to cause the processor 604 to associate a first amount of a first contrast agent with the zone, wherein the first contrast agent has a first imaging characteristic when imaged in the predetermined radiation environment and instructions 614 to cause the processor 604 to associate a second amount of a second contrast agent with the zone, wherein the second contrast agent has a second imaging characteristic when imaged in the predetermined radiation environment.

In some examples the first contrast agent provides a relatively larger change in image contrast level and the second contrast agent provides a relatively smaller change in image contrast level.

In some examples the first contrast agent and the second contrast agent may provide changes in contrast levels corresponding to different imaging methods. For example the first contrast agent may be used to provide an intended imaging characteristic (attenuation or contrast level) when the object is imaged using x-ray radiation and the second contrast agent may provide an intended imaging characteristic contrast level when imaged using MRI.

In some examples the instruction 606 may further comprise instructions to determine control instructions, which when executed, instruct an additive manufacturing apparatus to generate an object according to the object model data. In some examples the instructions 606 may further comprise instructions to execute the determined control instructions, thereby generating the object according to the derived object model data.

In some examples the instructions 606 may further comprise instructions to obtain an image indicative of attenuation of the generated object, for example as described in relation to block 308 of FIG. 3.

Examples in the present disclosure can be provided as methods, systems or machine-readable instructions, such as any combination of software, hardware, firmware or the like. Such machine-readable instructions may be included on a computer readable storage medium (including but not limited to disc storage, CD-ROM, optical storage, etc.) having computer readable program codes therein or thereon.

The present disclosure is described with reference to flow charts and/or block diagrams of the method, devices and systems according to examples of the present disclosure. Although the flow diagrams described above show a specific order of execution, the order of execution may differ from that which is depicted. Blocks described in relation to one flow chart may be combined with those of another flow chart. It shall be understood that each block in the flow charts and/or block diagrams, as well as combinations of the blocks in the flow charts and/or block diagrams can be realized by machine readable instructions.

The machine-readable instructions may, for example, be executed by a general purpose computer, a special purpose computer, an embedded processor or processors of other programmable data processing devices to realize the functions described in the description and diagrams. In particular, a processor or processing apparatus may execute the machine-readable instructions. Thus, functional modules of the apparatus and devices (such as the imaging characteristic module 404 and/or object model module 406) may be implemented by a processor executing machine readable instructions stored in a memory, or a processor operating in accordance with instructions embedded in logic circuitry. The term ‘processor’ is to be interpreted broadly to include a CPU, processing unit, ASIC, logic unit, or programmable gate array etc. The methods and functional modules may all be performed by a single processor or divided amongst several processors.

Such machine-readable instructions may also be stored in a computer readable storage that can guide the computer or other programmable data processing devices to operate in a specific mode.

Such machine-readable instructions may also be loaded onto a computer or other programmable data processing devices, so that the computer or other programmable data processing devices perform a series of operations to produce computer-implemented processing, thus the instructions executed on the computer or other programmable devices realize functions specified by block(s) in the flow charts and/or block diagrams.

Further, the teachings herein may be implemented in the form of a computer software product, the computer software product being stored in a storage medium and comprising a plurality of instructions for making a computer device implement the methods recited in the examples of the present disclosure.

While the method, apparatus and related aspects have been described with reference to certain examples, various modifications, changes, omissions, and substitutions can be made without departing from the spirit of the present disclosure. It is intended, therefore, that the method, apparatus and related aspects be limited only by the scope of the following claims and their equivalents. It should be noted that the above-mentioned examples illustrate rather than limit what is described herein, and that those skilled in the art will be able to design many alternative implementations without departing from the scope of the appended claims.

The word “comprising” does not exclude the presence of elements other than those listed in a claim, “a” or “an” does not exclude a plurality, and a single processor or other unit may fulfil the functions of several units recited in the claims.

The features of any dependent claim may be combined with the features of any of the independent claims or other dependent claims.

1. A method comprising:
defining, for an interior portion of an object to be generated in additive manufacturing, an imaging characteristic in a radiation environment; and
deriving object model data for generating the interior portion of the object by determining an amount of a first contrast agent and an amount of a second contrast agent to deposit such that the object generated according to the object model data comprises the defined imaging characteristic apparent in an image of at least a part of the object comprising the interior portion captured in the radiation environment,
wherein the first contrast agent and the second contrast agent have different imaging characteristics in the radiation environment.

2. A method as claimed in claim 1 wherein depositing the first contrast agent provides a relatively larger change in the imaging characteristic and depositing the second contrast agent provides a relatively smaller change in the imaging characteristic.

3. A method as claimed in claim 1 wherein the imaging characteristic is an attenuation.

4. A method as claimed in claim 3 wherein the attenuation is attenuation of x-ray radiation.

5. A method as claimed in claim 1 wherein the amount of the first contrast agent is selected to provide an intended x-ray attenuation and the amount of the second contrast agent is selected to provide an intended magnetic resonance imaging contrast.

6. A method as claimed in claim 1, comprising:
defining an imaging characteristic for each zone of a plurality of zones within the interior portion; and
wherein deriving the object model data comprises determining an amount of the first contrast agent and an amount of the second contrast agent for each zone.

7. A method as claimed in claim 1, wherein the object is a medical imaging phantom.

8. A method as claimed in claim 1 wherein the first and/or the second contrast agents comprise at least one of:
gadolinium;
a perfluorinated organic compound;
a metal salt;
a metallogenic compound;
a metal oxide; and
metal nanoparticles.

9. A method as claimed in claim 1, wherein deriving the object model data further comprises determining an amount of fusing agent to deposit to cause coalescence of a build material in additive manufacturing by absorbing energy applied to the build material.

10. A method as claimed in claim 1, further comprising:
generating the object in additive manufacturing according to the derived object model data.

11. An apparatus comprising processing circuitry, the processing circuitry comprising:
an imaging characteristic module to obtain, for an interior portion of an object to be generated in additive manufacturing, a predetermined image contrast level for when the object is imaged in a predetermined radiation condition; and
an object model module to derive object model data for use in generating the interior portion of the object, wherein the object model data specifies an amount of a first contrast agent and an amount of a second contrast agent to be deposited during generation of the object to provide the predetermined image contrast level apparent in an image of at least a part of the object comprising the interior portion captured when the object is imaged in the predetermined radiation condition;
wherein the first contrast agent has a first imaging characteristic and the second contrast agent has a second imaging characteristic when imaged in the predetermined radiation condition.

12. An apparatus as claimed in claim 11 wherein:
the radiation condition of the radiation environment describes a type of radiation and energy of the radiation.

13. An apparatus as claimed in claim 11, further comprising:
an additive manufacturing apparatus to generate the object based on the derived object model data.

14. A machine-readable medium comprising machine-readable instructions which, when executed by a processor, cause the processor to:
define, for an interior portion of an object to be generated in additive manufacturing, an image contrast level in a predetermined radiation environment; and
derive object model data for generating the interior portion of the object,
wherein deriving the object model data comprises, for each of a plurality of zones within the interior portion:
associating a first amount of a first contrast agent with the zone, wherein the first contrast agent has a first imaging characteristic when imaged in the predetermined radiation environment; and
associating a second amount of a second contrast agent with the zone, wherein the second contrast agent has a second imaging characteristic when imaged in the predetermined radiation environment.

15. A machine-readable medium as claimed in claim 14, wherein the first contrast agent provides a relatively larger change in image contrast level and the second contrast agent provides a relatively smaller change in image contrast level.
